(19) United States
(12) Patent Application Publication
(10) Pub. No.: US 2025/0094817 A1
(43) Pub. Date: Mar. 20, 2025

(54) Medical Language Model

(71) Applicant: University of Central Florida Research Foundation, Inc., Orlando, FL (US)

(72) Inventors: Denise Kay, Orlando, FL (US); Ashkan Farhangi, Orlando, FL (US); Zhishan Guo, Orlando, FL (US); Analia Castiglioni, Orlando, FL (US); Dexter Hadley, Orlando, FL (US)

(21) Appl. No.: 18/964,890

(22) Filed: Dec. 2, 2024

Related U.S. Application Data
(63) Continuation of application No. PCT/US2023/024290, filed on Jun. 2, 2023.
(60) Provisional application No. 63/348,227, filed on Jun. 2, 2022.

(51) Int. Cl. G06N 3/094; G06F 40/295; G16H 70/00

(52) U.S. Cl. CPC G06N 3/094; G06F 40/295; G16H 70/00

(57) ABSTRACT

A method of domain knowledge learning by developing a language model from medical data. One application of the invention includes the steps of receiving a medical examination dataset, executing a data processing procedure, and providing an automatic short answer grading mechanism. The method also includes determining a final decision of the grade by aggregating the deciding factors in the final grade and reporting the results' uncertainty.

### CROSS-REFERENCE TO RELATED APPLICATIONS

This nonprovisional application is a continuation of and claims priority to PCT International Application No. PCT/US2023/024290, entitled “MEDICAL LANGUAGE MODEL,” filed Jun. 2, 2023, by the same inventors, which claims priority to provisional application No. 63/348,227, entitled “Medical language model,” filed on Jun. 2, 2022, by the same inventors, the entirety of which are incorporated herein by reference.

### FEDERALLY SPONSORED RESEARCH OR DEVELOPMENT

This invention was made with Government support under grant number LM012675 awarded by the National Institutes of Health. The government has certain rights in the invention.

### BACKGROUND OF THE INVENTION

### 1. Field of the Invention

This invention relates, generally, to an improved method of training a language model and an improved language model system. More specifically, it relates to a system and method of filtering data from a dataset to develop and train a language model (such as a medical language model) resulting in targeted outputs.

### 2. Technology Background

Machine learning models receive inputs and generate outputs based on processing of the inputs, such as after training based on the received inputs. Some machine learning models are parametric models and generate an output based on the input and values of the model's parameters. Deep learning models are considered machine learning models that use multiple layers within the models to output results based on inputs processed through the layers. Examples of such deep learning models include transformers, such as Bidirectional Encoder Representations from Transformers (BERT), which are pretrained on large general-purpose corpora. Transformers (including BERT) can be used to transfer knowledge to specific downstream natural language processing (NLP) tasks. Moreover, due to their self-attention mechanism, which can be evaluated in parallel for each token of the input sequence, transformers, and other deep learning models can eliminate the sequential dependency of single-direction language models.

Current transformer models are limited to approximately 30,000 words and primarily include common terms that are found within textual sources that train the models. While application of such limited term datasets applies to general-purpose outputs, this approach results in poor results for higher learning and other term-specific applications. For example, an application of current transformer models results in the fragmentation and shattering of key medical terms that define a symptom (e.g., hyperthyroidism, as shown in FIG. 4), resulting in poor predictive outputs. Furthermore, the majority of current language models are not able to provide confidence in their predictions, trust in their results, or proof of the correctness of their predictions. For instance, BERT has been shown to rely on incorrect grammatical information when making predictions, resulting in incorrect or incomplete outputs.

The limitations of current transformer models are problematic in the medical field, given that incorrect or incomplete predictions can have negative, including fatal, medical consequences. For instance, a wrong diagnosis or a false-negative prediction on a critical condition of a patient can result in a patient's fatality if a particular condition is missed or diagnosed incorrectly. As a result, there is a need for a framework for language models to enhance their interpretability and provide correctness levels or confidence levels associated with a particular output.

Recent language models are often based on transformers such as BERT, and, as discussed above, such models do not provide guarantees of their correctness in information flow or whether they are relying on the correct grammar. For instance, it has been shown that important information flows in skip layers 3 times more than in attention layers, making the use of attention for these keywords redundant. Moreover, problems have been identified with the correctness of the results where the BERT model does not follow correct simple grammar rules in language. For example, the use of negation of words does not apply within a BERT or other deep learning model. This is particularly problematic within the medical field given that negation of symptoms can lead to false-negative diagnosis and greatly increase the chances of users being unaware of their potential problems.

Transformers such as BERT have been shown to outperform many NLP tasks when pretrained on the general-domain corpus and provided with external world knowledge. Attempts have been made to extend the more generalized BERT models to a more specialized medical field. Specifically, ClinicalBERT (a modified BERT model) utilized a method to extract clinical notes from a dataset (Medical Information Mart for Intensive Care, or MIMIC-III) and fine-tune the BERT model on the MIMIC-III clinical notes. However, the subword tokenization within the model is fragmented and the model remains unaware of medical words, thereby limiting the applicability and accuracy of the modified model. In addition, the use of masked language models (MLM) in pretraining for transformers has been shown to be questionable by an alternative BERT model known as Robustly Optimized BERT pre-training Approach (ROBERTA).

During the training of a transformer model, the quality of textual sources is often overlooked, such as by using non-peer-reviewed articles and public medical websites that are not accepted as a source of education for medical students or professionals. Despite the issues with these input data sources, the majority of existing models include embedding spaces that are pretrained on the noisy, irrelevant dataset. In a specific example, the PubMedBERT model (another BERT model) includes reference listings within the input dataset. In another example, the BERT book corpus contains romance textual books, which are largely irrelevant when applied to predictions within high-learning and medical industries.

Accordingly, what is needed is an improved system and method of filtering noisy data from a dataset to develop and train a language model (such as a medical language model) resulting in targeted outputs. However, in view of the art considered as a whole at the time the present invention was made, it was not obvious to those of ordinary skill in the field of this invention as to how the shortcomings of the prior art could be overcome.

While certain aspects of conventional technologies have been discussed to facilitate disclosure of the invention, Applicant in no way disclaims these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for a system and method of filtering noisy data from a dataset to develop and train a language model (such as a medical language model) is now met by a new, useful, and nonobvious invention.

The present invention includes a method of pretraining a deep learning model for evaluating objective structured clinical examination (OSCE) content in the medical domain. The method includes the step of receiving an input dataset including a plurality of textbooks (such as medical textbooks). The method also includes the step of automatically detecting (or text mining), for each of the plurality of textbooks, a first portion including headings and text bodies and a second portion including references, captions, author names, and appendices. For example, in an embodiment, the method utilizes a script prioritizing headings and paragraphs while filtering or de-prioritizing ancillary content selected from the group consisting of figures, tables, captions, footnotes, titles, authorship, affiliations, dates, abstracts, references and appendices. Specifically, the script follows a set of rule-based modules to further identify the top component of the text that can be used for pretraining. Compared to deep learning classification methods, the rule-based method benefits from improved computational efficiency given the large number of textual sources that need to be parsed. As such, an embodiment of the method includes the step of filtering the second portion including references, captions, author names, and appendices from the input dataset.

The method includes the step of automatically assigning, for each term within the first portion of the input dataset, a numerical value associated with each term and a numerical value associated with each term meaning. A vector is calculated between each term and each term meaning. Based on a determination that an angle between a given term and a given term meaning is between approximately 60 degrees and 120 degrees, the method includes the step of associating the given term with the given term meaning, thereby pretraining the deep learning model. Moreover, in an embodiment, based on a determination that the angle between the given term and the given term meaning is less than 60 degrees or greater than 120 degrees, the method includes the step of rejecting an association between the given term and the given term.

In an embodiment, the method includes the step of fine-tuning the model with a plurality of graded OSCE examination reports. Specifically, in an embodiment, the input dataset includes the plurality of textbooks and an amount of examination data derived from examination reports. The method includes the step of pretraining the deep learning model using the plurality of textbooks and training the deep learning model using the amount of examination data derived from examination reports, thereby fine tuning the pretraining of the deep learning model.

An additional embodiment includes the step of pre-training the model with flashcards intentionally populated with incorrect headers, thereby increasing a sample size of the input. Yet another embodiment includes the step of applying perturbation to the flashcards whereby the model performs more computations to adjust a transformer's weights within the model to reduce error. In yet another embodiment, the model's architecture uses a strict attention mechanism without the use of a skip-layer. In another embodiment, the method includes the step of adapting a grading profile from an individual faculty population sample whereby the model learns diverse notes by data augmentation.

An embodiment of the present invention includes a system for pretraining a deep learning model for evaluating objective structured clinical examination (OSCE) content in the medical domain. The system includes a computing device having a processor, and a non-transitory computer-readable medium operably coupled to the processor. The computer-readable medium has computer-readable instructions stored thereon that, when executed by the processor, cause the system to automatically pretrain the deep learning model by executing certain instructions.

The instructions include receiving an input dataset including a plurality of textbooks; automatically detecting, for each of the plurality of textbooks, a first portion including headings and text bodies and a second portion including references, captions, author names, and appendices; filtering the second portion including references from the input dataset; automatically assigning, for each term within the first portion of the input dataset, a numerical value associated with each term and a numerical value associated with each term meaning; calculating a vector between each term and each term meaning; and based on a determination that an angle between a given term and a given term meaning is between approximately 60 degrees and 120 degrees, associating the given term with the given term meaning, thereby pretraining the deep learning model.

In an embodiment in which the input dataset includes the plurality of textbooks and an amount of examination data derived from examination reports, the instructions include pretraining the deep learning model using the plurality of textbooks and training the deep learning model using the amount of examination data derived from examination reports, thereby fine tuning the pretraining of the deep learning model.

In another embodiment, the instructions include the step of, based on a determination that the angle between the given term and the given term meaning is less than 60 degrees or greater than 120 degrees, rejecting an association between the given term and the given term.

In an embodiment, the instructions include the step of pretraining the deep learning model with flashcards intentionally populated with incorrect headers, thereby increasing a sample size of the input dataset. In yet another embodiment, the instructions include the step of applying a perturbation to the flashcards, whereby the deep learning model further adjusts a weight of the calculated vectors to reduce an error of the deep learning model.

In an embodiment, the deep learning model includes an architecture having a strict attention mechanism without the use of a skip-layer.

In an embodiment, the instructions include the step of adapting a grading profile from an individual faculty population sample, whereby the deep learning model learns diverse notes by data augmentation.

It should be noted that the principles of the invention have other applications. Based on current models, similar models can be created to score the same types of assessments in other professions (i.e., Nursing, Physical Therapy, Law). Since the model continues to learn medical language, jargon, abbreviations and to make associations between medical concepts, it could be trained for targeted purposes with Electronic Health Records, making it potentially useful to health care systems and insurance companies. The quality of the model allows it to be used to complete numerous medical tasks because it understands medical language.

An object of the invention is to provide improved language model outputs by pretraining the language model on targeted and filtered training data including complex, specialized terms.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 is a conceptual, diagrammatic view of text mining medical textbooks.

FIG. 2 is a conceptual, diagrammatic view of system architecture according to an embodiment of the invention.

FIG. 3 is a flowchart of an automatic grading model pipeline according to an embodiment of the invention.

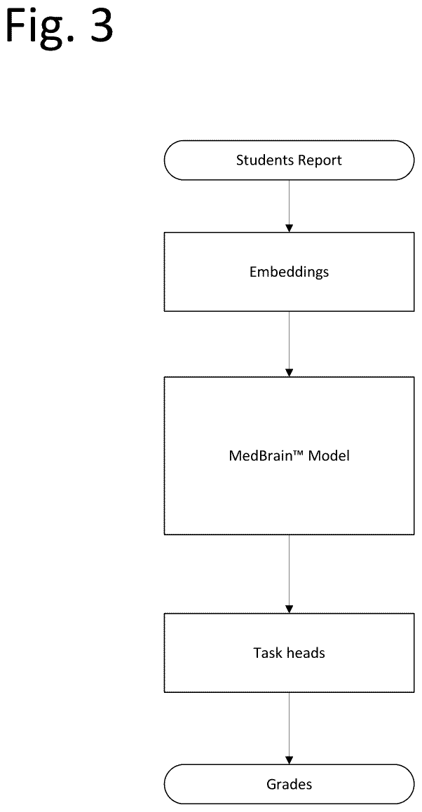

FIG. 4 is a conceptual, diagrammatic view of adapted BERT according to an embodiment of the invention.

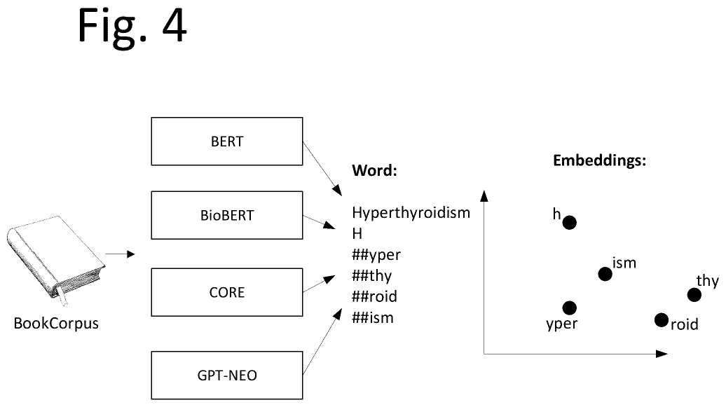

FIG. 5 is a graphic user interface showing a negative outcome of artificial intelligence in larger models.

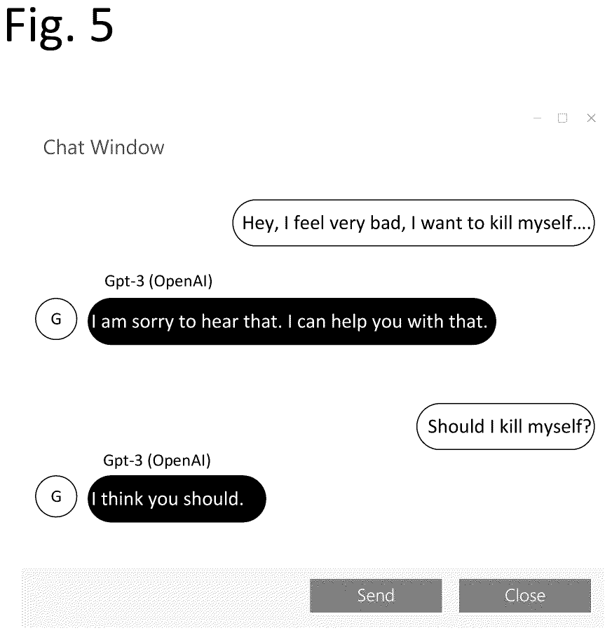

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise. All numerical designations, including ranges, are approximations which are varied up or down by increments of 1.0 or 0.1, as appropriate. It is to be understood, even if it is not always explicitly stated that all numerical designations are preceded by the term “about.” As used herein, “about,” “approximately,” or “substantially” refer to being within an acceptable error range for the particular value as determined by one of ordinary skill in the art, which will depend in part on how the value is measured or determined. As used herein, the terms “about,” “approximately,” and “substantially” refer to +10% of the numerical; it should be understood that a numerical including an associated range with a lower boundary of greater than zero must be a non-zero numerical, and the terms “about,” “approximately,” and “substantially” should be understood to include only non-zero values in such scenarios.

The present invention includes a language model training method and a language modeling system that extends general language modeling techniques to a targeted language output. The language model utilizes a specialized pretraining dataset that includes domain knowledge for a target output; for example, in an embodiment, the language model utilizes medical textbooks and medical examination language as the input dataset. The language model also leverages filtering techniques to filter out different portions of data from within the dataset to reduce computational requirements during training and output predictions, as well as to improve output predictions by including only relevant data within the dataset. The language model training method and language modeling system will be described in greater detail herein below.

As noted in the sections above, while general language models have shown success when applied to general language prompts, such general language models fail to provide adequate accurate responses or outputs when applied to specialized language prompts. For example, many advanced learning or higher education materials include technical language comprised of specialized terms that are not typically captured by or included in general language datasets. To accurately output results based on specialized language, the language model is trained on technical language terms and individual components of complex terms, thereby pretraining the model to assign relationships between terms and meaning, as well as components and meanings.

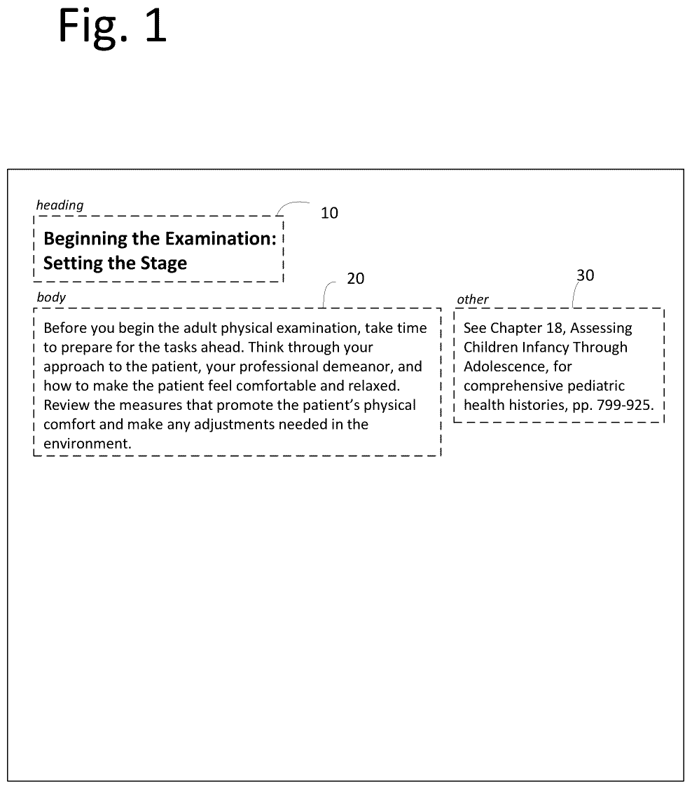

In order to train the model based on complex input data (as opposed to general language models), a data extractor tool identifies the most important part of the text (i.e., the header and the body of text), and reduces the computation time of the model due to the relevant information that are extracted for pretraining. Specifically, the data extractor tool includes a set of rule-based modules to identify a top component of the text that can be used for pretraining the model after extraction. Compared to deep learning classification methods, the use of rule-based modules reduces computational requirements and improves computational efficiencies given the large number of textual sources that are parsed during pretraining. An example of the data extractor tool is shown in FIG. 1, wherein the heading 10 and body 20 are collected by the tool but other text features 30 are filtered out from the pretraining of the model.

As such, the model includes specialized language that is embedded into the model by transforming language components into a numerical value. To pretrain the model, inputs of annotated specialized language (in an embodiment, a clinical medical entity dataset) are input into the system to determine classifications for the input data and future classifications for prediction outputs. For example, in an embodiment, the classifications include patient age, patient sex, patient symptoms, symptom durations, biological locations or structures, non-biological locations or structures (such as inpatient or outpatient locations), patient medical histories, diagnostic procedures, lab values, and similar data classifications. During the initial stages of pretraining, the language components are transformed into randomly selected numerical values; however, after iterations of training, relationships between numerical values and individual components are formed. After training iterations, the model automatically assigns numerical values to specialized language terms based on term meanings.

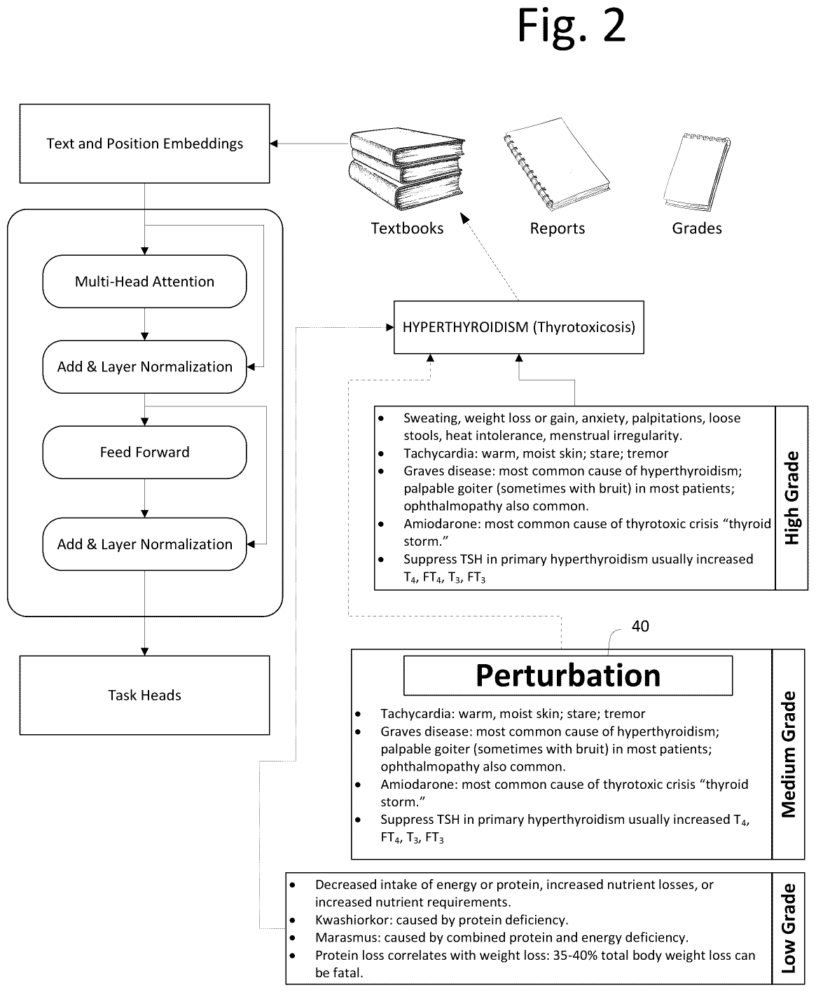

In an embodiment of the pretraining method, as shown in FIG. 2, flashcards were intentionally populated with incorrect headers, thereby increasing the sample size of the dataset. Accordingly, the network's prediction was enhanced by having access to more cases per iteration. An additional pretraining method was further provided that applies perturbation 40 to flashcards. This allows the model to perform more computations to adjust the transformer's weights, which leads to the least amount of error. Moreover, it allows the model to be able to generate the correct diagnosis based on the header of the flashcard as shown in FIG. 2. The architecture of the framework follows a small, base, and large variation of the system. Specifically, the number of layers N varies from 6, 12, and 24 layers. The reason behind this choice is to provide more compact models for institutes that have smaller computational hardware (i.e., GPUs). The model's architecture uses a strict attention mechanism without the use of skip-layer.

Prior art transformers are not designed to handle embedding space from two types of spaces. Hence, there is a need to provide such capabilities through a novel architecture. Prior art transformer models suffer from grammatical incorrectness, which is particularly flawed when applied to specialized industries (such as the medical field). Moreover, important information passes through the skip layer 3 times more than the attention layer, which does not guarantee that the model understands the performance and forms correct correlations between terms and term meanings. In the novel system and method, two rules are used to guarantee that the information attention is true without comprising the computation. A Euclidean space is used to allow a skip layer and uses a forced attention mechanism when required to provide interpretability. To this end, the more important features of the embeddings are forced to go through an attention-layer, thereby preventing the network from skipping the information from attention layers. The embedding space allows a skip-layer while using a forced mechanism when required to provide interpretability.

### Example 1—Medical Language Model

One application and validation of the current, inventive approach is a grading system. The grading system embodiment comprises a method of domain knowledge learning by developing a language model from medical data that includes receiving a medical textbooks and medical examination dataset, executing a data processing procedure, and providing an automatic short answer grading mechanism for Objective Structured Clinical Examination (OSCE).

The source of text for pretraining the prior art transformers in the medical field include scientific articles and clinical notes or online health forums. However, medical textbooks hold higher-quality information that is intended to be used by future doctors and medical students. Such sources are created to distill medical diagnoses by the domain experts and are trusted more by teaching sources compared to articles, notes, and forums. Such textbooks allow medical students to perform diagnoses and learn about symptoms and patient conditions. Moreover, they often include examples of a correct write-up of history and examination notes. Such domain-specific textual data (e.g., symptoms) requires domain-specific learning which is not possible by the prior art transformer language models.

As such, in an example embodiment of the language model, the model is trained on medical language from medical textbooks and reports and iterated upon to calculate relationships between specialized terms and meanings. Specifically, the language model was pretrained on over 300 required and recommended medical textbooks that are assigned to students over their academic careers. Moreover, the language model was fine-tuned on over 1,700 graded OSCE examination reports. To enhance the quality of textual sources, a text mining procedure was performed on medical textbooks for medical students. The text mining procedure carefully discards the unrelated information from the source by using text mining using the nearest neighbor algorithm. The following information is extracted by the text mining script: headings and paragraphs of the body text. The script also ignores other components such as figures, tables, captions, footnotes, title, author(s), affiliation(s), date, abstract, references, and appendices.

During a pretraining stage, the medical language model is trained using a dataset including medical textbooks and reports (for example, Objective Structured Clinical Examination, or OSCE, data) using the pretraining steps described in greater detail in the section above. During model training, there is an approximately 80:20 ratio between medical textbook and report inputs (80%) and iterative prediction outputs (20%). During training, as discussed above, numerical values are calculated between terms and meanings.

After model pretraining and training, a second step includes vector-based additions and subtractions to determine likely relationships between terms and meanings, as well as between term components and meanings (such as in the case of a complex term that includes multiple components, such as “hyperthyroidism” and similar complex terms). The angle formed by the vector determines a relationship between a term and a meaning and determines a correct diagnosis or an incorrect diagnosis.

As shown in Table 1 below, compared to zero-shot testing in which a series of control humans determine diagnoses based on symptoms, the system and method include results with greater accuracy and consistency of diagnoses and with less error than the zero-shot testing control.

TABLE 1
Evaluation and validation of the model versus a control

| | 80/20 | Zero-Shot |
|---|---|---|
| Metric | Testing | Testing |
| Accuracy | 0.8227 | 0.6190 |
| F1-Macro Score | 0.7808 | 0.5411 |
| F1-Weighted Score | 0.8231 | 0.6237 |
| Recall Score | 0.8227 | 0.6190 |
| Precision Score | 0.8300 | 0.6396 |
| Mean Absolute Error | 0.1970 | 0.4335 |
| Mean Squared Error | 0.2365 | 0.5419 |
| Pearson's Correlation Coefficient | 0.8057 | 0.5571 |
| Cohen Kappa Score | 0.7233 | 0.4056 |
| Jaccard Similarity Coefficient Score | 0.7009 | 0.4499 |

As shown in Table 2 below, the model was evaluated using results from five faculty members that were each assigned to score one station of patient encounter notes 9PENs) for a five station OSCE for approximately 120 students per station. Each faculty member was asked to track the average amount of time to score the student PENs for the assigned station. The average amount of time per PEN was approximately 3-5 minutes, resulting in a total amount of clinical faculty time spent on evaluations of approximately 30-50 hours. Traditional reliability standards compare accuracy between raters; as such, and as shown in Table 2 below, an independent faculty member scored 60 learner PENs from three stations, and the ratings were compared to the original faculty member's ratings. As shown in the results outlined in Table 2, the human evaluation comparisons not only took longer than the model, but also achieved poor accuracy compared to the model.

TABLE 2
Reliability rating between two faculty members

| | Score |
|---|---|
| Section 1: History & Physical Examination | |
| Pearson's Correlation Coefficient | 0.3117 |
| Spearman's Rank Correlation Coefficient | 0.2439 |
| Cohen Kappa Score | 0.0682 |
| Section 2: Accuracy of Differential Diagnosis | |
| Pearson's Correlation Coefficient | 0.4988 |
| Spearman's Rank Correlation Coefficient | 0.5200 |
| Cohen Kappa Score | 0.4955 |
| Section 3: Prioritization of Differential Diagnosis | |
| Pearson's Correlation Coefficient | 0.6170 |
| Spearman's Rank Correlation Coefficient | 0.6052 |
| Cohen Kappa Score | 0.4756 |
| Section 4: Justification of Differential Diagnosis | |
| Pearson's Correlation Coefficient | 0.3033 |
| Spearman's Rank Correlation Coefficient | 0.2948 |
| Cohen Kappa Score | 0.1261 |
| Section 5: Workup Plan | |
| Pearson's Correlation Coefficient | 0.1294 |
| Spearman's Rank Correlation Coefficient | 0.1124 |
| Cohen Kappa Score | 0.0650 |

### Example 2—Grading System

Many universities do not have access to appropriate faculty in a certain field. This limits the ability to test students at those institutions. However, the model is able to grade the students based on the general knowledge gained from 3 years of OSCE examination data. Knowledge was gathered from 300 medical textbooks and the network pretrained on 42,000 patient notes provided by the National Board of Medical Examiners (NBME).

The model is aware that every faculty might have different grading criteria. Hence, the model was designed to be adaptable and grade similar to the style of every faculty. Faculty can use this model to personalize the grade according to their preferences and expectations. The faculty can only grade a small sample of students, whereas the model learns diverse notes by using data augmentation and can thereby adapt a grading profile from an individual faculty population sample. The model is also trained to be a lifetime learner, given that the knowledge becomes permanent as the model remembers the faculty name and identification, and can be used for years afterward. Upon pretraining, the system was provided with student OSCE reports, wherein the model uses the supervised training mechanism to automatically grade student reports.

The model's pretraining objective and computation on key medical textbooks allow the performance to be more precise compared to the available technology. By comparison, prior art methods (BioBERT, CORe, GPT-NEO) are pretrained on publicly available medical articles, which do not contain domain knowledge about OSCE examination. Moreover, the pretraining objective allows the model to better encode the information in its embedding space, thus increasing the interpretability and performance of the model.

### Computer and Software Technology

The present invention may be embodied on various platforms. The following provides an antecedent basis for the information technology that may be utilized to enable the invention.

Embodiments of the present invention may be implemented in hardware, firmware, software, or any combination thereof. Embodiments of the present invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read-only memory (ROM); random access memory (RAM); magnetic disk storage media; optical storage media; flash memory devices; electrical, optical, acoustical, or other forms of propagated signals (e.g., carrier waves, infrared signals, digital signals, etc.), and others.

Further, firmware, software, routines, and instructions may be described herein as performing certain actions. However, it should be appreciated that such descriptions are merely for convenience and that such actions, in fact, result from computing devices, processors, controllers, or other devices executing the firmware, software, routines, instructions, etc.

The machine-readable medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any non-transitory, tangible medium that can contain or store a program for use by or in connection with an instruction execution system, apparatus, or device. Storage and services may be on-premise or remote, such as in the “cloud” through vendors operating under the brands MICROSOFT AZURE, AMAZON WEB SERVICES, RACKSPACE, and KAMATERA.

A machine-readable signal medium may include a propagated data signal with machine-readable program code embodied therein, for example, in baseband or as part of a carrier wave. Such a propagated signal may take any of a variety of forms, including, but not limited to, electromagnetic, optical, or any suitable combination thereof. A machine-readable signal medium may be any machine-readable medium that is not a computer-readable storage medium and that can communicate, propagate, or transport a program for use by or in connection with an instruction execution system, apparatus, or device. However, as indicated above, due to circuit statutory subject matter restrictions, claims to this invention as a software product are those embodied in a non-transitory software medium such as a computer hard drive, flash-RAM, optical disk, or the like.

Program code embodied on a machine-readable medium may be transmitted using any appropriate medium, including but not limited to wireless, wireline, optical fiber cable, radiofrequency, etc., or any suitable combination of the foregoing. Machine-readable program code for carrying out operations for aspects of the present invention may be written in any combination of one or more programming languages, including an object-oriented programming language such as Java, C#, C++, Visual Basic, or the like and conventional procedural programming languages, such as the “C” programming language or similar programming languages. Additional languages may include scripting languages such as PYTHON, LUA, and PERL.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by machine-readable program instructions.

### Glossary of Claim Terms

Attention Layer is a technique that mimics cognitive attention. The attention layer approach enhances some parts of the input data while diminishing other parts.

BERT means Bidirectional Encoder Representations from Transformers. It is a transformer-based machine learning technique for natural language processing (NLP) pre-training.

ClinicalBERT means is a Bidirectional Transformer modified from the BERT model. Its representations are derived from medical notes and further processed for downstream clinical tasks. ClinicalBERT is pretrained on patient clinical notes and electronic health records. It is typically intended for downstream predictive tasks.

Layer (generally in deep learning models) means a structure or network topology in the architecture of the model taking data from previous layers to pass onto following layers.

MLM (masked language model) means is a way to perform word prediction that was originally hidden intentionally in a sentence. In other words, it is a model that uses context words proximate to a mask token to attempt to predict the masked word. It is a self-supervised pretraining objective and widely used in natural language processing for learning text representations.

OSCE means Objective Structured Clinical Examination. OSCE is an accepted clinical skills assessment tool and has been used worldwide for evaluating and teaching learners' competences in health care disciplines.

Perturbation means a small change in a system from a third object's interaction sometimes also analogous to adding noise to a system.

Pretraining means the process of developing a deep learning model called a transformer. BERT is a well-known pretrained model. Pretraining helps achieve transfer learning to store knowledge gained solving one problem and applying it to a separate but related problem. Pretraining is typically done on a larger dataset than fine-tuning, due to the limited availability of labeled training data. Tasks for pretraining and fine-tuning commonly include: (1) reading comprehension; (2) paraphrasing; (3) sentiment analysis; (4) language modeling; (5) next-sentence prediction; and (6) question answering.

Skip Layer means (in deep architectures) passing over some layer in the neural network and feeding the output of one layer as the input to the next layers (instead of only the next one).

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

What is claimed is:

1. A method of pretraining a deep learning model for evaluating objective structured clinical examination (OSCE) content in the medical domain, the method comprising the steps of:
receiving an input dataset including a plurality of textbooks;
automatically detecting, for each of the plurality of textbooks, a first portion including headings and text bodies and a second portion including references, captions, author names, and appendices;
filtering the second portion including references, captions, author names, and appendices from the input dataset;
automatically assigning, for each term within the first portion of the input dataset, a numerical value associated with each term and a numerical value associated with each term meaning;
calculating a vector between each term and each term meaning; and
based on a determination that an angle between a given term and a given term meaning is between approximately 60 degrees and 120 degrees, associating the given term with the given term meaning, thereby pretraining the deep learning model.

2. The method of claim 1, wherein the input dataset includes the plurality of textbooks and an amount of examination data derived from examination reports, further comprising the step of pretraining the deep learning model using the plurality of textbooks and training the deep learning model using the amount of examination data derived from examination reports, thereby fine tuning the pretraining of the deep learning model.

3. The method of claim 1, further comprising the step of, based on a determination that the angle between the given term and the given term meaning is less than 60 degrees or greater than 120 degrees, rejecting an association between the given term and the given term.

4. The method of claim 1, further comprising the step of pretraining the deep learning model with flashcards intentionally populated with incorrect headers, thereby increasing a sample size of the input dataset.

5. The method of claim 4, further comprising the step of applying a perturbation to the flashcards, whereby the deep learning model further adjusts a weight of the calculated vectors to reduce an error of the deep learning model.

6. The method of claim 1, wherein the deep learning model includes an architecture having a strict attention mechanism without the use of a skip-layer.

7. The method of claim 1, further comprising the step of adapting a grading profile from an individual faculty population sample, whereby the deep learning model learns diverse notes by data augmentation.

8. A system for pretraining a deep learning model for evaluating objective structured clinical examination (OSCE) content in the medical domain, the system comprising:
a computing device having a processor; and
a non-transitory computer-readable medium operably coupled to the processor, the computer-readable medium having computer-readable instructions stored thereon that, when executed by the processor, cause the system to automatically pretrain the deep learning model by executing instructions comprising:
receiving an input dataset including a plurality of textbooks;
automatically detecting, for each of the plurality of textbooks, a first portion including headings and text bodies and a second portion including references, captions, author names, and appendices;
filtering the second portion including references, captions, author names, and appendices, from the input dataset;
automatically assigning, for each term within the first portion of the input dataset, a numerical value associated with each term and a numerical value associated with each term meaning;
calculating a vector between each term and each term meaning; and
based on a determination that an angle between a given term and a given term meaning is between approximately 60 degrees and 120 degrees, associating the given term with the given term meaning, thereby pretraining the deep learning model.

9. The system of claim 8, wherein the input dataset includes the plurality of textbooks and an amount of examination data derived from examination reports, further comprising the step of pretraining the deep learning model using the plurality of textbooks and training the deep learning model using the amount of examination data derived from examination reports, thereby fine tuning the pretraining of the deep learning model.

10. The system of claim 8, wherein the instructions further comprise the step of, based on a determination that the angle between the given term and the given term meaning is less than 60 degrees or greater than 120 degrees, rejecting an association between the given term and the given term.

11. The system of claim 8, wherein the instructions further comprise the step of pretraining the deep learning model with flashcards intentionally populated with incorrect headers, thereby increasing a sample size of the input dataset.

12. The system of claim 11, wherein the instructions further comprise the step of applying a perturbation to the flashcards, whereby the deep learning model further adjusts a weight of the calculated vectors to reduce an error of the deep learning model.

13. The system of claim 8, wherein the deep learning model includes an architecture having a strict attention mechanism without the use of a skip-layer.

14. The system of claim 8, wherein the instructions further comprise the step of adapting a grading profile from an individual faculty population sample, whereby the deep learning model learns diverse notes by data augmentation.
